(19) United States
(12) Patent Application Publication
(10) Pub. No.: US 2006/0229538 A1
(43) Pub. Date: Oct. 12, 2006

(54) Rehabilitation device

(76) Inventors: Steven B. Schmidt, Sacramento, CA (US); Stacey R. Vincilione, Rocklin, CA (US)

Correspondence Address: GROSS & ASSOCIATES, 8541 DOMINIQUE COURT, FAIR OAKS, CA, 95628, US

(21) Appl. No.: 11/101,050

(22) Filed: Apr. 6, 2005

(51) Int. Cl. A61F 5/00

(52) U.S. Cl. 602/6

(57) ABSTRACT

The present inventions provides a device for aiding a user in recovering from an incision or injury, comprising a member having a bottom surface, a first body being disposed adjacent to the bottom surface of the member; a second body being disposed adjacent to the member; and, a channel formed between the member, the first body and the second body.

### BACKGROUND OF THE INVENTION

1. Field of Invention

The present invention relates to a patient rehabilitation, comfort and injury protection device and more particularly to a pillow.

2. Description of Related Art

Conventional devices exist that provide protection for an injury or surgical incision while the healing process proceeds. Some of these conventional devices have an elevated area over the incision or wound to protect it from contamination and provide a form of cushioning against contact. Further, often these incisions extend through muscle tissue. Doctors generally desire the patient to use these muscles after surgery in an effort to heal the cut muscle tissue. However, it is usually very painful to use these muscles soon after an incision, such as an abdominal incision. To ease the pain, the patient generally wants to hold or press against an area surrounding the cut muscle tissue. Currently, no conventional devices exist that assist the user with applying this pressure to the area surrounding an incision. If a patient uses a common pillow by holding and pressing it over the incision, the pillow will undesirably impact or touch the incision area increasing the pain. Still further, simply using just the hands is not comfortable to the patient. Accordingly, there is a need for a device that addresses these and other issues as well as the shortcomings of conventional devices.

### SUMMARY OF INVENTION

It is an object of the present invention to overcome the drawbacks and shortcomings of conventional recovery surgery pillow devices. The present invention allows a user to apply pressure on both sides of an incision without contacting the incision. The present invention provides this additional comfort by incorporating a channel or gap, which raises the pillow off a user thereby reducing the contact of the pillow with sensitive incision or injury areas. Further, in other exemplary embodiments, this present invention employs straps to hold the device on the user allowing the user the free use of hands and ensuring the device is always available to calm painful muscle spasms that frequently and randomly arise after surgery. Additionally, the present invention, in other exemplary embodiments can be wrapped around a user without the straps to provide the same comforts.

The present invention provides a more efficient rehabilitation device than conventional devices in that it can be used for multiple applications, such as for example chest and abdominal post-operative applications as well as other major muscle area such as the legs and arms. Further, this present invention provides comfort to painful rehabilitating process helping a user return to normal life. Moreover, many conventional rehabilitation devices are limited in their versatility; in particular, these conventional devices have one operational position thereby limiting their applicability by a user in different situations. The present invention however, provides a rehabilitation device that, in some embodiments, has at least an extended position as well as a collapsed position and can be easily rotated by a user. The multiple positions allow a user to readily adjust the dimension of the device as well as use the device in various situations. For example, the present invention can be used while sitting with the device in the collapsed position or lying down with the device in the extended position.

In the application of chest and abdominal incisions, this invention provides the benefit of promoting breathing and coughing to avoid sedentary complications after surgery, such as atelectasis, poor circulation and pneumonia, thereby increasing mobility to further assure successful recovery and decreased recovery time. This is accomplished by providing a user more comfort when chest or abdominal muscles contract, reducing pain to the user and hence the user more willing to breathe and cough. The above mentioned straps on the present invention for chest and/or abdominal incisions provides for increased success by maintaining the pillow in an easily usable position for activities, such as walking, sleeping, and driving a car. In the case of other major muscle surgery, doctors often require patients to exercise the major muscles that have been operated on with activities such as physical movement to avoid sedentary complications. The present invention also aids a user in these activities to increase recovery success and reduce recovery time.

The present invention provides a rehabilitation device comprising of a member having a bottom surface, a first body being disposed adjacent to the bottom surface of the member, a second body being disposed adjacent to the member; and, a channel formed between the member, the first body and the second body.

Additionally, the present invention provides a curative post-operative pillow comprising of a member, a first body pivotably attached to the member; and, a second body attached to the member, wherein a gap is formed between the member, and the first and second bodies.

Further, the present invention provides a post-operative device comprising of a band securable by a user; and, first and second bodies being disposed adjacent to the band, wherein a channel is a channel is formed between the first and second bodies.

These and other features and advantages of this invention are described in, or are apparent from, the following detailed description of various exemplary embodiments of the devices and methods according to this invention.

### BRIEF DESCRIPTION OF FIGURES

Various exemplary embodiments of this invention will be described in detail, with reference to the following Figures, wherein:

FIG. 1 is a top front perspective view of a device according to this invention, shown in a first position;

FIG. 2 is a bottom front perspective view of the device of FIG. 1;

FIG. 3 is a top front perspective view of the device of FIG. 1, shown in a second position, which is a different operation position from the first position;

FIG. 4 is a perspective view of a user illustrating an exemplary use of the device of FIG. 1;

FIG. 5 is a perspective view of an alternative embodiment of a device made in accordance with this invention;

FIG. 6 is a perspective view of another alternative embodiment of a device made in accordance with this invention;

FIG. 7 is a perspective view of yet another alternative embodiment of a device made in accordance with this invention;

FIG. 8 is a perspective view of another alternative embodiment of a device made in accordance with this invention;

FIG. 9 is a perspective view of yet another alternative embodiment of a device made in accordance with this invention;

FIG. 10 is an exploded perspective view of another alternative embodiment of a device made in accordance with this invention;

FIG. 11 is a cross-sectional view taken along line 11-11 in FIG. 10;

FIG. 12 is a perspective view of yet another alternative embodiment of a device made in accordance with this invention; and,

FIG. 13 is a perspective view of yet another alternative embodiment of a device made in accordance with this invention.

### DETAILED DESCRIPTION OF INVENTION

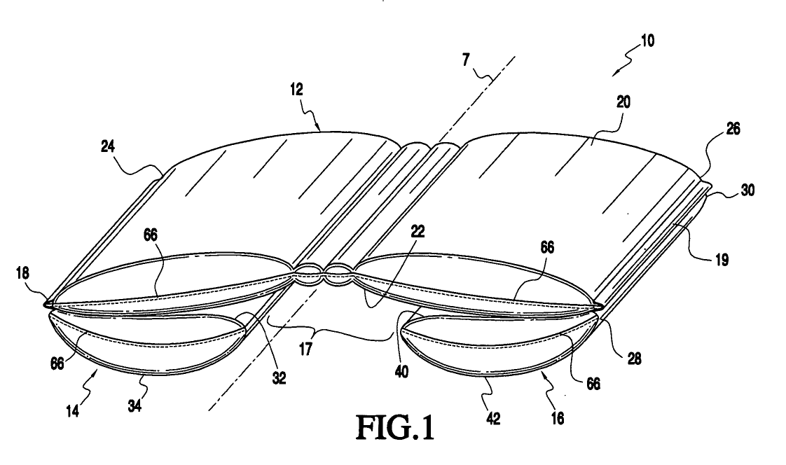

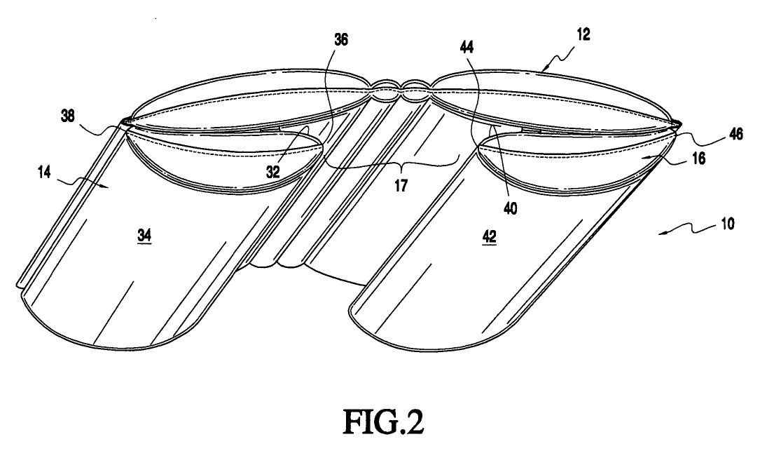

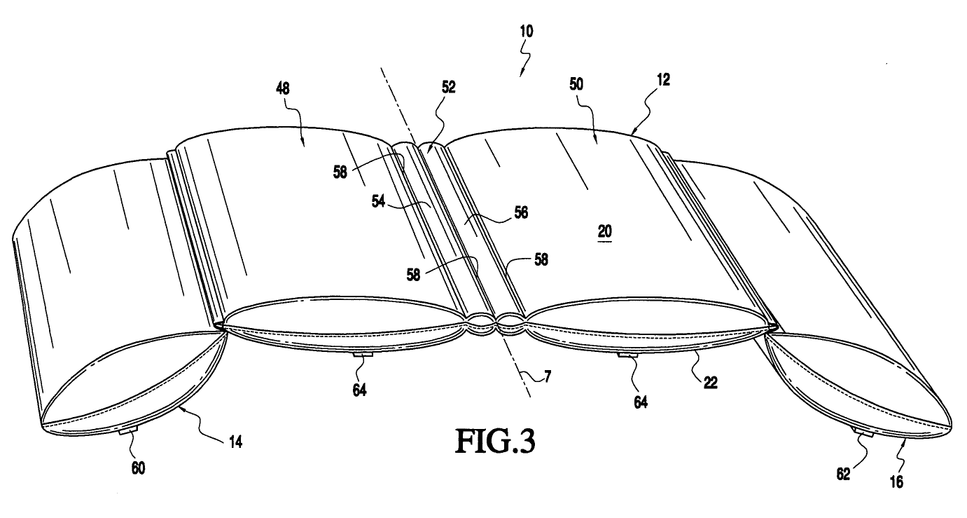

FIGS. 1 and 2 are perspective views of a device 10, which is an exemplary embodiment of a rehabilitation device made in accordance with this invention. The device 10 may be used for a variety of applications including, but not limited to, patient rehabilitation, comfort and injury protection. The device 10 may also be broadly considered to be a pillow. The device 10 includes a main member 12, a first body 14, and a second body 16. A channel or gap 17 is defined between the main member 12, and the first and second bodies 14 and 16. The device 10 is shown in a first position in FIGS. 1 and 2. This first position may be referred to as a collapsed position. The device 10 is shown in a second position in FIG. 3. The second position may be referred to as an extended position. In either position, the device 10 may be utilized by a user to be placed over an injury or incision area. It is preferred that the channel 17 be aligned with the injury or incision area such that the device 10 is not directly on top of or pressing against the injury or incision area.

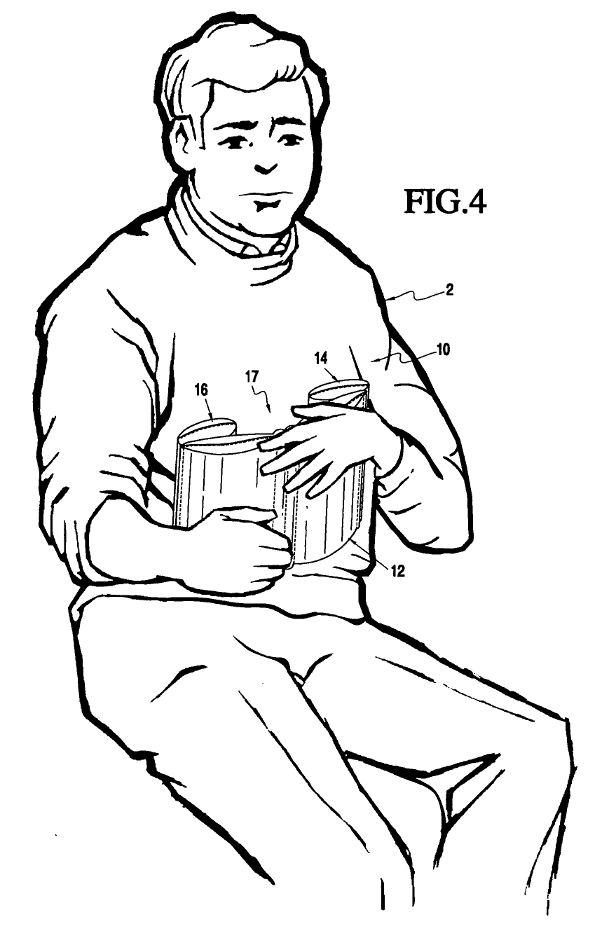

FIG. 4, shows an exemplary application of the device 10, wherein a user 2 is holding the device 10 against his abdominal area. The user in this exemplary illustration has an abdominal injury or incision (not shown) and the channel 17 of the device 10 is disposed just above the injury or incision. Often times injuries and incisions affect the muscle tissue. Generally, as part of rehabilitation, doctors instruct the user (patient) to cough, breath deep and ambulate so as to use these muscles to increase recovery success. These movements and activities help prevent sedentary complications, such as atelectasis, blood circulation and pneumonia. In FIG. 4, it is the abdominal muscles that have the incision. In other cases, the incision or injury may affect other areas of a patient, such as the chest. The device 10 is positioned over the incision, as shown, with the channel 17 directly above the incision and the first and second bodies 14 and 16 on either side thereof. The user 2 is able to pull against the device 10 with his hands/arms against the body around the incision area. By pulling the device 10 against the incision area, the user is better able to utilize the injured muscles as well as ease the pain caused by such muscle utilization. In many cases the injury or incision is closed using staples as sutures, which cause discomfort to apply direct pressure to the incision area. Because of the channel 17, the pressure will not be directly applied to the incision area. It should be appreciated that the device 10 could also be used in a similar fashion in the second or extended position. The extended position may be desirable to a user when the user is lying on her side in a bed or when the user is in a car, for example.

In this exemplary embodiment, as shown in FIGS. 1 through 4, the first and second bodies 14 and 16 are pivotably attached to the member 12 at pivot connections 18 and 19, respectively. The first and second bodies 14 and 16 are pivotable about the pivot connection 18 and 19, respectively, so as to be positioned in the collapsed position or first position, as shown in FIG. 1, or to be positioned in the extended position, as shown in FIG. 3, or to be positioned anywhere between the collapsed and extended positions. The user will selectively determine the position the device 10 should be in for the particular application. Further, one of the first and second bodies 14 and 16 can selectively be in the extended configuration while the other is in the collapsed configuration.

The main member 12 and the first and second bodies 14 and 16 are preferably constructed of a foam material. It should be appreciated that other materials could be utilized in other embodiments, such as any type of pillow filling material or the like. The surface material of the device 10 is preferred to be any material that is smooth and soft to the touch. However, it should be appreciated that certain areas of the device 10, in other various exemplary embodiments, are made of or incorporate materials that facilitate fastening the first and second bodies 14 and 16 and other components to the main member in various positions, such as a material that allows hooks of a hook and loop fastener to attach to it. The main member 12 and the first and second bodies 14 and 16 are preferably covered with a soft material. It is preferred that the outer material be smooth. In other various exemplary embodiments the material can be rougher like terri-cloth for example. Further, it is should be appreciated that the material is preferably constructed out of a hypo-allergenic material.

Referring to FIGS. 1, 2 and 3, it will be appreciated that the main member 12 has a first major surface 20 and a second major surface 22. The first and second major surfaces 20 and 22 may be thought of as top and bottom surfaces of the main member 12, respectively. The main member 12 also has first and second sides 24 and 26. It is preferred that the first and second major surfaces 20 and 22 extend between the first and second sides 24 and 26. It is further preferred that the first and second major surfaces 20 and 22 extend the entire length of the device 10, which is defined between a first end 28 and a second end 30 of the device. It should be appreciated that in other exemplary embodiments, the main member has an undulating surface along the length of the device (not shown). In this exemplary embodiment the length of first and second sides 14 and 16 are the same as main member 12. It should be appreciated that in other various exemplary embodiments the length of first and second sides may be longer or shorter then the length of the main member. Further, in this exemplary embodiment the length of first and second sides 14 and 16 are the same. It should be appreciated that in other various exemplary embodiments the length of first and second sides may be longer or shorter then each other.

The first body 14 has a first major surface 32 and a second major surface 34. The first and second major surfaces 32 and 34 may be thought of as top and bottom surfaces of the first body 14, respectively. The first body 14 also has first and second sides 36 and 38. It is preferred that the first and second major surfaces 34 and 36 extend between the first and second sides 36 and 38. It is further preferred that the first and second major surfaces 20 and 22 extend the entire length of the device 10.

The second body 16 is preferred to be similar in design to the first body 14. Particularly, the second body 16 also has first and second major surfaces 40 and 42, as well as first and second sides 44 and 46.

As indicated above, in this exemplary embodiment, the first and second bodies 14 and 16 are pivotably attached to the main member 12. Particularly, it is preferred that the second side 38 of the first body 14 is pivotably attached to the first side 24 of the main member 12 along the pivot connection 18. Likewise, it is preferred that the second side 46 of the second body 16 is pivotably attached to the second side 26 of the main member 12 along the pivot connection 19. In this exemplary embodiment, the pivot connections 18 and 19 are made via sewing the first and second bodies 14 and 16 to the main member 12. It is preferred that the pivot connections 18 and 19 extend the entire length of the device 10. However, it should be appreciated that in other various exemplary embodiments, the pivot connections do not extend the entire length and/or each include multiple pivot connections. It should be appreciated that in other various exemplary embodiments, the first and second bodies may be attached to the main member by other methods such as but not limited to zippers, hook and loop fasteners, buttons, snaps, etc.

The main member 12 also preferably includes three portions, a first portion 48, a second portion 50 and a middle portion 52, as clearly shown in FIG. 3. The middle portion 52 is preferably disposed between the first and second portions 48 and 50. It is preferred that the first and second portions 48 and 50 have substantially elliptical cross-sectional shapes. However, it should be appreciated that in other exemplary embodiments the cross-sections are shapes other than elliptical. Any shape may be utilized in these other embodiments.

It is further preferred that the middle portion 52 of the member 12 extend along a centerline 7 of the device. Yet further, it is preferred that the middle portion 52 be of a substantially thinner dimension than the central portions of the first and second portions 48 and 50. The middle portion 52 also preferably includes first and second section 54 and 56. The sections 54 and 56 are preferably defined or formed by, but not limited to seams or stitching 58 in the main member 12. The dimensional width of the entire middle portion 52 is preferably substantially less than the dimensional widths of the first and second portions 48 and 50. With this arrangement, the middle portion 52 will tend to be stiffer than the first or second portions 48 and 50. By being relatively more stiff than the rest of the main member 12, as well as the first and second bodies 14 and 16, the middle portion 52 provides some additional support along the centerline 7 of the device 10. Additional support along the centerline 7 is desirous because the centerline 7 of the device 10 will usually be disposed above and parallel with an incision of the user 2.

It is preferred that the first and second bodies 14 and 16 have an elliptical cross-section. However, it should be appreciated that in other exemplary embodiments the cross-sections are shapes other than elliptical. Any shape may be utilized in these other embodiments.

It is also preferred that the first and second bodies 14 and 16 have fasteners 60 and 62, so that non-fixed sides of the bodies 14 and 16 can temporarily attach to mating fasteners 64 on the main member 12 when the device 10 is in the collapsed position. In this way, the device 10 will remain in the collapsed position until the fasteners are disengaged and then the user 2 could open the device 10 to the extended position shown in FIG. 3. In this embodiment, the fasteners 60, 62 and 64 are hook and loop fasteners. However, it should be appreciated that a variety of other fasteners could be utilized, such as but not limited to, zippers, buttons, snaps, tie strings, etc. It should be appreciated that the fasteners 60, 62 and 64 are optional.

As discussed above, it is preferred that the main member 12 and the first and second bodies 14 and 16 be made of pillow type material with an outer covering enclosing the material. In this exemplary embodiment, the covering is sealed by stitching seams 66. However, it should be appreciated that in other various exemplary embodiments a variety of other types of enclosing the material could be utilized, such as but not limited to, zippers, buttons, snaps, tie strings, etc.

Referring again to FIGS. 1, 2 and 3, the process of making this exemplary embodiment starts from three separate foam pillows wherein, the main member 12 and the first and second bodies 14 and 16 are formed by stitching and joined at the pivot connections 18 and 19. However, it should be appreciated that in other various exemplary embodiments a variety of other means of connecting the main member and the first and second bodies could be utilized, such as but not limited to, zippers, buttons, snaps, tie strings, etc. Further, it should be appreciated that in other various exemplary embodiments the process of making the device could start from a single foam pillow wherein, the main member and the first and second bodies are formed or separated from one another by stitching at the pivot connections.

Referring again to FIG. 1, the device 10 can be manufactured to any desired length, width and thickness of the user. The preferred range for the length of the main member 12 is 10 to 20 centimeters. The preferred range for the width of the main member 12 is 11 to 37 centimeters. The preferred range for the thickness of the main member 12 is one to five centimeters. In this exemplary embodiment the length of the main member 12 is 20 centimeters, the width of the main member 12 is 24.5 centimeters and the thickness of the main member 12 is three centimeters.

The preferred range for the length of the first and second members 14 and 16 is 10 to 20 centimeters. The preferred range for the width of the first and second members 14 and 16 is five to 15 centimeters. The preferred range for the thickness of the first and second members 14 and 16 is one to five centimeters. In this exemplary embodiment the length of the first and second members 14 and 16 is 20 centimeters, the width of the first and second members 14 and 16 is 10 centimeters and the thickness of the first and second members 14 and 16 is three centimeters.

Further, the preferred range for the length of the channel 17 is 10 to 20 centimeters. The preferred range for the width of the channel 17 is one to seven centimeters. The preferred range for the height of the channel 17 is one to five centimeters. In this exemplary embodiment the length of the channel 17 is 10 centimeters, the width of the channel 17 is four centimeters and the height of the channel 17 is three centimeters.

The channel 17 of device 10 of this present invention aides the user 2 in the reduction of any further pain or trauma to stapled incision/injury area by providing a means of protection while permitting comfortable freedom to move or ease of movement. This protection is provided by the channel 17 raising the main member 12 off of a user. The device further aides the user 2 to follow surgeon's instruction to prevent post-operation complications caused by being sedentary. Further, the device 10 allows the user 2 to cough, breath, move, and walk without much additional supervision, per a doctor's instructions to increase activity post-surgery.

FIG. 5 displays a device 10, which is an alternative embodiment of a pillow made in accordance with the present invention. The device 10 provides additional stability and stiffness so that a user can pull with greater force against the device 10. The device 10 is similar to the device 10 described above and includes similar features, such as for example a main member 112, first and second bodies 114 and 116, and a channel 117. The main member 112 includes first and second portions 148 and 150, as well as a middle portion 152. One of the differences in this embodiment is that the middle portion 152 includes a receiving chamber 180. The receiving chamber 180 is operably configured to receive an insert member 182. The insert member 182 is preferably a substantially rigid member. It is further preferred that the insert member 182 have a dimension that is operably configured such that the insert member 182 can easily be inserted into the receiving chamber 180. It should be appreciated that the insert member can be made out of a variety of materials and does not have to be completely solid.

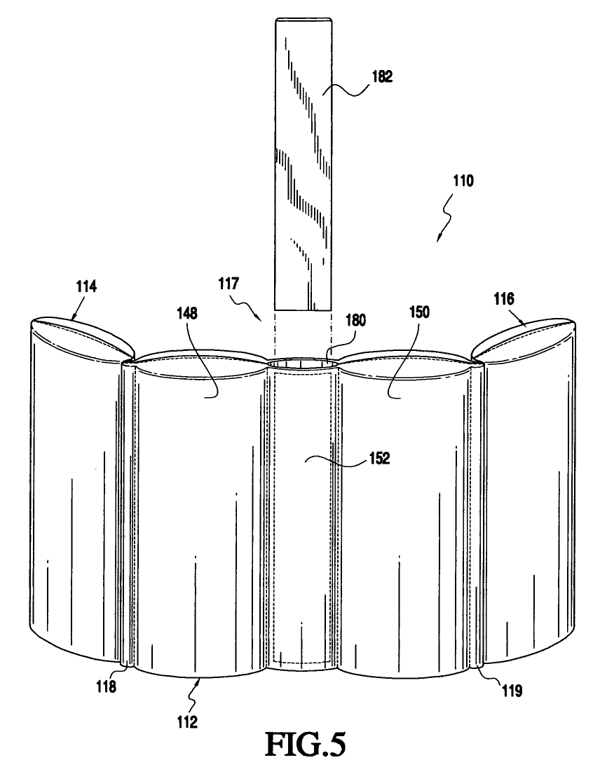

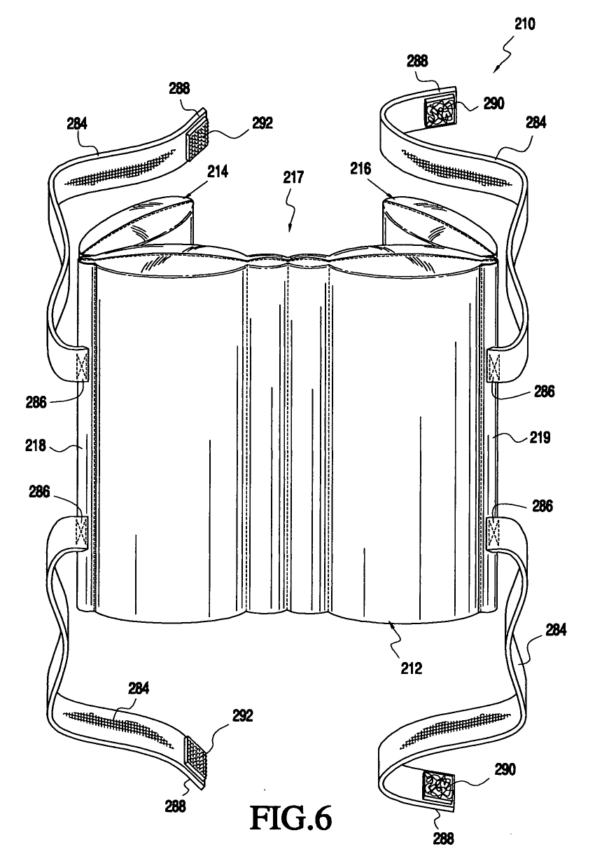

FIG. 6 displays a device 210, which is an alternative embodiment of a pillow made in accordance with the present invention. The device 210 provides means for a user to temporarily attach the device to himself/herself. The device 210 is similar to the device 10 described above and includes similar features, such as for example a main member 212, first and second bodies 214 and 216, and a channel 217. The device 210 also has pivot connections 218 and 219.

One of the differences in this embodiment is that the device 210 further includes a plurality of straps 284. In this embodiment, there are four straps 284. The straps 284 are attached to the device 210. Preferably, the straps are attached to or adjacent to the main member 212. It is preferred that two of the four straps 284 be attached to a side of the main member 212 that is opposing a side to which the other two straps 284 are attached. In this manner, the opposing straps 284 are operably configured to reach around a user to attach to one another. Each of the straps 284 has an attached end 286 and a free end 288. The attached ends 286 are fixed to the main member 212. It is preferred that the attached ends 286 be stitched to the main member 212, as shown for example. However, it should be appreciated that in other various exemplary embodiments the attached ends could be connected to the main member by a variety of other methods, such as but not limited to, hook and loop fasteners, buttons, snaps, tie strings, etc. The free ends 288 have fasteners 290 and 292. In this embodiment, the fasteners 290 and 292 are hook and loop fasteners. However, it should be appreciated that a variety of other fasteners could be utilized, such as but not limited to, buttons, snaps, tie strings, etc. It should also be appreciated that in other various exemplary embodiments, the attached ends of the straps are not permanently attached to the device. Rather, the attachment points, with d-rings for example, not shown, are disposed at various locations on the device and the attached ends of the straps engage the attachment points to secure the strap to the device 210. It should be further appreciated that in other various exemplary embodiments, there is only one strap, which can take on a variety of forms. The single strap can simply be a non-stretchable strap that extends from one side of the device around the user to the other opposing side of the device. Further, the single strap can, in other embodiments, be an elastic band that is attached to the device and wraps around the user. Still further, it should be appreciated that there may be any number of straps to assist in keeping the device adjacent to a user.

FIG. 7 displays a device 310, which is an alternative embodiment of a pillow made in accordance with the present invention. The device 310 provides means for a user to temporarily attach the device to himself/herself, such as around the neck. The device 310 is similar to the device 10 described above and includes similar features, such as for example a main member 312, first and second bodies 314 and 316, and a channel 317. The device 310 also includes pivot connections 318 and 319.

One of the differences in this embodiment is that the device 310 further includes a plurality of straps 384. In this embodiment, there are two straps 384. The straps 384 are attached to the device 310. Preferably, the straps are attached to or adjacent to the main member 312. It is preferred that the straps 384 be attached to an end of the main member 312. In this manner, the straps 384 are operably configured to reach around the neck of a user and attach to one another, or further, the straps 384 are operably configured to be attachable to other items, such as for example but not limited to, a shoulder strap in a vehicle. Each of the straps 384 has an attached end 386 and a free end 388. The attached ends 386 are fixed to the main member 312. It is preferred that the attached ends 386 be stitched. However, it should be appreciated that in other various exemplary embodiments the attached ends could be connected to the main member by a variety of other methods, such as but not limited to, hook and loop fasteners, buttons, snaps, tie strings, etc. The free ends 388 have fasteners 390 and 392, and fasteners 394 are disposed along the length of the straps 384. In this embodiment, the fasteners 390, 392 and 394 are hook and loop type fasteners. However, it should be appreciated that a variety of other fasteners could be utilized, such as but not limited to, buttons, snaps, tie strings, etc. It should also be appreciated that in other various exemplary embodiments, the attached ends of the straps are not permanently attached to the device 310. Rather, the attachment points, with d-rings for example, not shown, are disposed at various locations on the device and the attached ends of the straps engage the attachment points to secure the strap to the device 310. It should be further appreciated that in other various exemplary embodiments, there is only one strap, which can take on a variety of forms. The single strap can simply be a non-stretchable strap that extends from an end of the device around the user and/or another item and back to the same end of the device. Further, the single strap can, in other embodiments, be an elastic band that is attached to the device and wraps around the user and/or another item. Still further, it should be appreciated that there may be any number of straps to assist in keeping the device adjacent to a user.

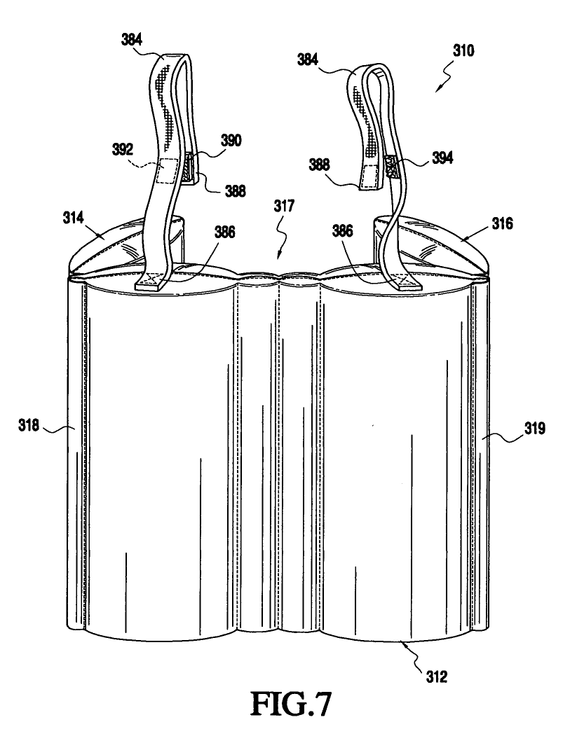

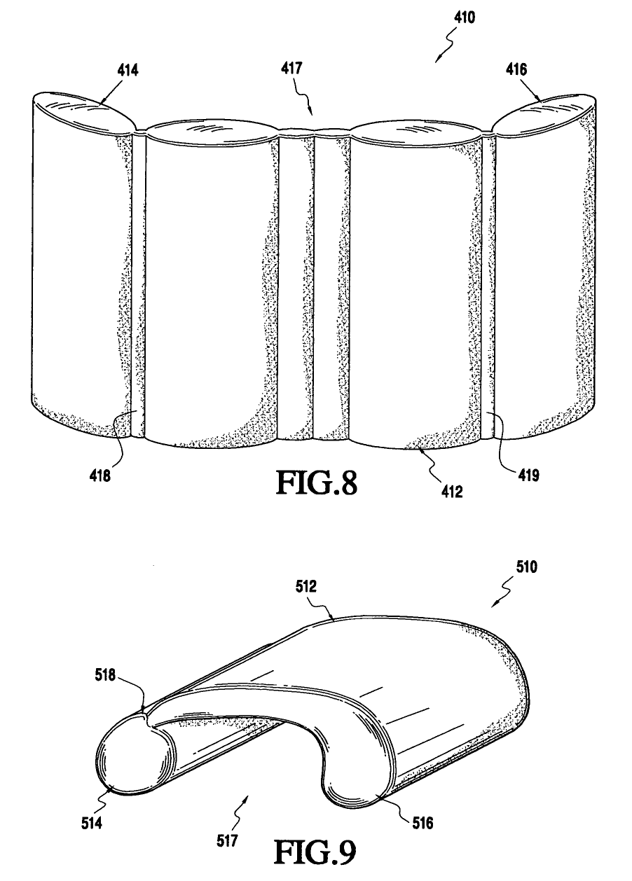

FIG. 8 displays a device 410, which is an alternative embodiment of a pillow made in accordance with the present invention. The device 410 illustrates an exemplary embodiment wherein the entire device is one unitary member. The device 410 is similar to the device 10 described above and includes similar features, such as for example a main member 412, first and second bodies 414 and 416, and a channel 417. Pivot connections 418 and 419 pivotally connect the first and second bodies 414 and 416 to the main body 412. One of the differences in this embodiment is that the device 410 is a unitary device. Particularly, the main member 412 and the first and second bodies 414 and 416 are a single unitary piece. The main member 412 separates the first and second bodies 414 and 416 by transition portions. The transition portions are the pivot connections 418 and 419. In this embodiment, the device is made of a foam material. It preferred that the foam material is shaped so as to take on the preferred cross-sections for the various elements as discussed above for the device 10, and as shown.

FIG. 9 displays a device 510, which is an alternative embodiment of a pillow made in accordance with the present invention. The device 510 illustrates an exemplary embodiment wherein at least two components of the device are unitary. The device 510 is similar to the device 10 described above and includes similar features, such as for example a main member 512, first and second bodies 514 and 516, and a channel 517. One of the differences in this embodiment is that the main member 512 is integral with the second body 516. It should be appreciated that the main member 512 is in other embodiments integral with the first body rather than the second body. Another difference in this embodiment is that only the first body 514 is pivotably connected at pivot connection 518. In other words, only the first body 514, and not the second body 516, can pivot to the extended configuration. With this embodiment, the costs manufacturing of the device may be reduced while still providing versatility of the device to the user in that the user can flip the device so as to extend the device in a direction or to the side the user desires.

The main member 512 is connected to the first body 514 along connection 518. It is preferred that the connection be made by stitching. It should be appreciated that in other various exemplary embodiments, the first body may be attached to the main member by other methods such as but not limited to hook and loop fasteners, buttons, snaps, etc.

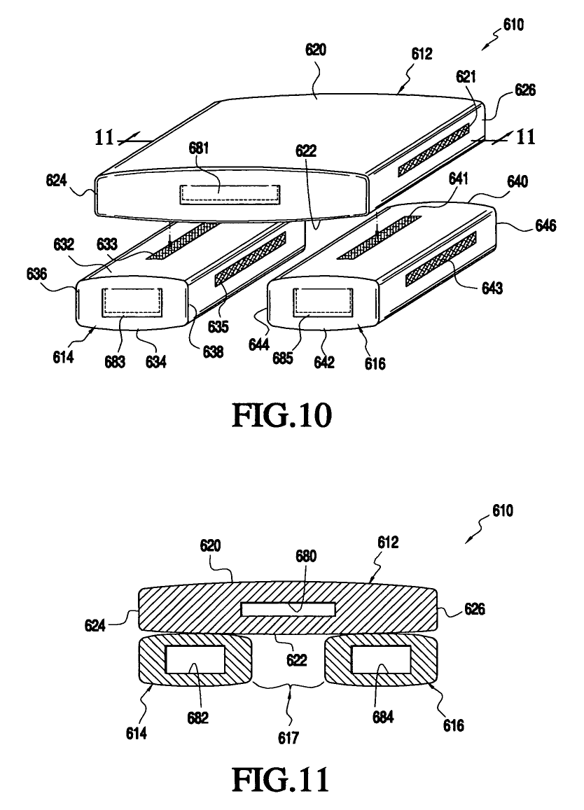

FIGS. 10 and 11 display a device 610, which is an alternative embodiment of a pillow made in accordance with the present invention. The device 610 is similar to the device 10 described above and includes similar features, such as for example a main member 612, first and second bodies 614 and 616, and a channel 617. The main member 612 has a top 620, a bottom 622, a first side 624 and a second side 626. The first and second bodies 614 and 616 each have a top 632, 640; a bottom 634, 642; a first side 636, 644; and, a second side 638, 646 respectively.

One of the differences in this embodiment is in the manner that the first and second bodies 614 and 616 are attached to the main member 612. This different attachment manner provides additional versatility for a user including the ability to vary the size of channel 617. Similar to the device 10, the first and second bodies 614 and 616 are attachable to the main member 612, such that the device 612 has at least two positions. FIG. 10 is an exploded perspective view of the device 610 in the first position and is similar the open position of the device as described above with reference to FIGS. 1 and 2. The second position, which is not shown, is the expanded position and is similar to the position of the device as describe above with reference to FIG. 3. In the second position, the main member 612 has the first body 614 attached to main member side 624 and second body 616 attached to main member side 626. The preferred method for attaching first and second bodies 614 and 616 is by hook and loop fasteners as shown in FIG. 10. However, it should be appreciated that in other various exemplary embodiments other fastening methods common to the art could be employed. FIG. 11, is a cross-sectional view of the device 610 along line 11-11.

In this embodiment, the first and second bodies 614 and 616 are removably attached to main member 612. It should be appreciated that the first and second bodies 614 and 616 are attached to the main member 612 by means of fasteners 633 and 641, respectively, as shown is FIG. 10, which mate with corresponding fasteners on the bottom surface 622 of the main member 612, not shown. The fasteners 633 and 641 are preferably disposed on the top surfaces 632 and 640 of the first and second bodies 614 and 616, respectively. It should be appreciated that in other exemplary embodiments the fasteners may substantially cover the top surfaces to allow the user to make an adjustment in the channel. It is preferred that the fasteners 633 and 641, as well as the mating fasteners, not shown, on the main member 612 be hook and loop type fasteners. It should be appreciated that in other various exemplary embodiments the member can be attached to the first and second bodies by other means common or known to one skilled in the art to allow the first and/or the second bodies to be removable from the main member. Further, it should be appreciated that in other exemplary embodiments, there are multiple fasteners attaching the bodies to the main member.

The first and second bodies 614 and 616 also include fasteners 635 and 643, respectively, on at least one side. In this embodiment, the fasteners 635 and 643 are disposed on the second sides 638 and 646. This is the second position and is not shown. Further, the main member 612 includes fasteners 621 on its side surfaces 624 and 626, as shown in FIG. 10. To attain the second position or extended position of the device 610, (not shown), the first and second bodies 614 and 616 are detached from the bottom surface 622 of the main member 612 and reattached to the first and second sides 624 and 626, respectively of the main member via the above noted fasteners 635, 643 and 621. It is preferred that the fasteners 635 and 643, as well as the mating fasteners 621, on the main member 612, be hook and loop type fasteners. It should be appreciated that in other various exemplary embodiments the member can be attached to the first and second bodies by other means common or known to one skilled in the art to allow the first and/or the second bodies to be removable from the main member. Further, it should be appreciated that in other exemplary embodiments, there are multiple fasteners attaching the bodies to the main member. Additionally, in should be appreciate that in other exemplary embodiments the first and second bodies can be attached to the main member such that the second sides of the first and second bodies attach to the bottom surface of the main member. This would allow the channel to have a deeper depth.

In this exemplary embodiment the main member 612 and the first and second bodies 614 and 616 are shown as elongated rectangular shapes. However, it should be appreciated that in other various exemplary embodiments, these components can be a variety of shapes such as tapered along its length and they do not all have to have the same or similar shapes.

FIG. 11 is a cross-sectional view of the device 610 taken along line 11-11 shown in FIG. 10. It is preferred that the main member 612 and the first and second bodies 614 and 616, each have an internal chamber 680, 682 and 684, respectively.

It is preferred that the chambers 680, 682 and 684 extend along a significant portion of the length of the respective components, such as for example 95 percent of the length. However, it should be appreciated that the lengths can extend to any percentage of the lengths of the respective components. It should further be appreciated that the chambers 680, 682 and 684 can have different lengths from one another.

The chamber 680 is similar to the chamber 180 of the device 110 disclosed above with reference to FIG. 5. Similar to the chamber 180, the chamber 680 is operably configured to receive an insert member, not shown. The insert member is preferably a stiffener. It should be appreciated that the stiffener is optional based on the user's preference.

The chambers 682 and 684 of the first and second bodies 614 and 616 are operably configured to receive insert members (not shown) as well. However, it is preferred that these chambers be of a sufficient volume capacity to receive other medical devices, such as hot and cold packs. The chambers 680, 682 and 684 have an access opening at one end of the device 610 and it is preferred that each of the openings have a cover 681, 683 and 685, respectively, as depicted in FIGS. 10-11. It is preferred that the covers 681, 683 and 685 be flaps and constructed out of the same material as the device 610. The covers 680, 682 and 684 are held closed by hook and loop fasteners. It should be appreciated that the covers can be held closed by other fasteners or combinations thereof.

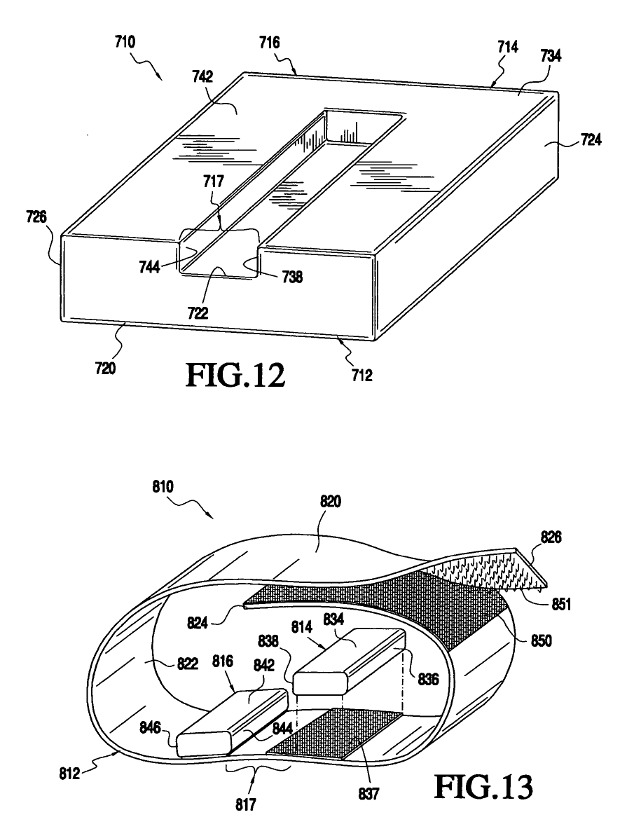

FIG. 12 displays a device 710, which is an alternative embodiment of a pillow made in accordance with the present invention. The device 710 is similar to the device 10 described above and includes similar features, such as for example a main member 712, first and second bodies 714 and 716, and a channel 717.

One of the differences in this embodiment is that the first and second bodies 714 and 716 are fixed to the main member 712. This different attachment manner provides a channel 717 having a fixed channel width. The first and second bodies 714 and 716 in this embodiment are integral to the main member 712. It should be appreciated that in other various exemplary embodiments, first and second bodies 714 and 716 can be separate bodies permanently attached to the main member 712. Additionally, a difference in this embodiment is that the length of channel 717 is not equal to the length of the main member 712 so that the channel does not extend the length of the device 710. In this exemplary embodiment the channel 717 is closed at one end and open at the opposing end.

FIG. 13 displays a device 810, which is an alternative embodiment of a pillow made in accordance with the present invention. The device 810 is similar to the device 10 described above and includes similar features, such as for example a main member 812, first and second bodies 814 and 816, and a channel 817.

One of the differences in this embodiment is in the manner that the first and second bodies 814 and 816 are attached to the main member 812. While second body 816 is fixed to the main member 812, first body 814 is adjustable in its position thus changing the width of the channel 817. The first body 814 is removable from the main body 812 and can be reattached to the main body 812 in either closer or further away from second body 816 as desired by the user. In this exemplary embodiment, first body 814 is attached by hook and loop fasteners 837 and 838. It should be appreciated that in other various exemplary embodiments first body could be removably attached to the main member 812 by other methods then described above, such as buttons, snaps, stitching, etc. Further, it should be appreciated that in other various exemplary embodiments first body could be permanently attached to the main member 812 and the second body be removably attached to the main member 812. In this exemplary embodiment second member 816 is attached to the main member by a means of stitching. In other various exemplary examples the second body could be attached to the main member by other methods such as but not limited to snaps, hook and loop fastener, zipper, etc. This different attachment manner provides additional versatility for a user including the ability to vary the size of channel 817. Further, it should be appreciated that in other various exemplary embodiments the first and second bodies could be displaced on the main member such that the first and second bodies are rotated 90 degrees from the position shown in FIG. 13, such that the channel is substantially parallel to the length of the main member. This would allow the user to apply the device for surgical incisions that are horizontal on the body.

Another difference is that the main member 812 is elongated along the first and second major surfaces 820 and 822. Similar to the device 10, it should be appreciated that the main member 812 has a first major surface 820 and a second major surface 822. The first and second major surfaces 820 and 822 are elongated as shown in FIG. 13, such that the main member 812 will wrap around a user allowing the device 810 to be attached to the user. The main member 812 may be referred to as a band. In this embodiment, the first and second major surfaces 820 and 822 are fastened together by the use of hook and loop fasteners 850 and 851. It should be appreciated that in other various exemplary embodiments, the first and second major surfaces 820 and 822 could be fastened together by other methods common in the art such as but not limited to zippers, string ties, buttons etc. It should be appreciated that in other various exemplary examples, the main member is a continuous band. In this exemplary embodiment, main member 812 is made out of an elastic material. It should be appreciated that main member 812 could be constructed out of a foam or other soft material, or a combination of materials.

It will be appreciated that any reference above to an attachment method, such as stitching or a fastener of any type, such as hook and loop fasteners, or the like, is not intended to limit the invention to such a particular fastener or attachment means, unless specifically noted, and that one skilled in the art will recognize that other types of known fasteners can be used for the respective particular applications.

It should be appreciated that in other various exemplary embodiments, devices made in accordance with this invention may incorporate some or all or a combination of the features of the various embodiments described and suggested above.

While this invention has been described in conjunction with the specific embodiments outlined above, it is evident that many alternatives, modifications and variations will be apparent to those skilled in the art. Accordingly, the preferred embodiments of the invention, as set forth above, are intended to be illustrative, not limiting. Various changes may be made without departing from the spirit and scope of this invention.

What is claimed is:

1. A rehabilitation device comprising:
a member having a bottom surface;
a first body being disposed adjacent to the bottom surface of the member;
a second body being disposed adjacent to the member; and,
a channel formed between the member, the first body and the second body.

2. A device, as recited in claim 1, wherein the channel extends from a first side to a second side of the member.

3. A device, as recited in claim 1, wherein a channel formed between the member, the first body and the second body, wherein the channel extends partially from a first side to a second side of the member.

4. A device, as recited in claim 1, wherein at least one of the first and second bodies is pivotably attached to the member.

5. A device, as recited in claim 1, wherein at least one of the first and second bodies is integral to the member.

6. A device, as recited in claim 1, wherein at least one of the first and second bodies is removable from the member.

7. A device, as recited in claim 1, wherein at least one of the first and second bodies is disposable in one of a plurality of locations adjacent to the member to vary the size of the channel.

8. A device, as recited in claim 1, wherein at least one of the first and second bodies includes a compartment to receive other devices.

9. A device, as recited in claim 1, wherein the member includes a compartment operably configured to receive a stiffener.

10. A device, as recited in claim 1, further comprising at least one strap operably configured to attach the device to a user

11. A device, as recited in claim 1, wherein the member further includes opposing first and second ends, the first and second ends extend outward such that the first and second ends are operably configured to be securable to a user.

12. A device, as recited in claim 1, wherein the first body is substantially disposed beneath the member.

13. A device, as recited in claim 1, wherein at least one of the member, the first body or the second body is a pillow.

14. A curative post-operative pillow comprising:
a member;
a first body pivotably attached to the member; and,
a second body attached to the member, wherein a gap is formed between the member, and the first and second bodies.

15. A device, as recited in claim 14, wherein the second body is attached at an opposing end of the member from the first body.

16. A device, as recited in claim 14, wherein the first and second bodies are pivotably attached to the main member.

17. A device, as recited in claim 14, wherein at least one of the first and second bodies is integral to the member.

18. A device, as recited in claim 14, wherein second body is removably attached to the member.

19. A device, as recited in claim 14, wherein the member includes a compartment operably configured to receive a stiffener.

20. A device, as recited in claim 14, wherein the gap extends partially from a first end to a second end of the member.

21. A device, as recited in claim 14, wherein at least one of the member, the first body or the second body is a pillow.

22. A post-operative device comprising:
a band securable by a user; and,
first and second bodies being disposed adjacent to the band, wherein a channel is a channel is formed between the first and second bodies.

23. A device, as recited in claim 22, wherein the band includes first and second ends operably configured to overlap.

24. A device, as recited in claim 22, wherein the channel is substantially parallel with to the first and second ends.

25. A device, as recited in claim 22, wherein the channel extends from the entire length of the band.

26. A device, as recited in claim 22, wherein at least one of the first and second bodies are removably attached to the band.

27. A device, as recited in claim 22, wherein at least one of the first and second bodies is integral with the band.

28. A device, as recited in claim 22, wherein at least one of the first and second bodies is disposed at a plurality of locations on the band to vary a width of the channel.

29. A device, as recited in claim 22, wherein the band is elastic.

30. A device, as recited in claim 22, wherein the band is non-continuous.
